(19) United States
(12) Patent Application Publication
(10) Pub. No.: US 2021/0364485 A1
(43) Pub. Date: Nov. 25, 2021

(54) AIR QUALITY MONITOR

(71) Applicant: Ademco Inc., Golden Valley, MN (US)

(72) Inventors: Petr Fadrny, Blansko (CZ); Frantisek Sofka, Hrusovany nad Jevisovkou (CZ)

(21) Appl. No.: 16/881,913

(22) Filed: May 22, 2020

(51) Int. Cl. G01N 33/00

(52) U.S. Cl. CPC G01N 33/0032

(57) ABSTRACT

An air quality monitor includes a housing defining an inlet and an outlet, a fan, and one or more sensors disposed in the housing. The fan is configured to transport air along a nonlinear flow path from the inlet of the housing to the outlet of the housing. The one or more sensors are arranged along the nonlinear flow path and include a first sensor positioned upstream of the fan and a second sensor positioned downstream of the fan. The first sensor is configured to sense a first parameter of the air and the second sensor is configured to sense a second parameter of the air.

### TECHNICAL FIELD

The disclosure relates to air quality monitors.

### BACKGROUND

An air quality monitor may monitor air quality of air in a room by intermittently or continuously taking samples of the air and measuring certain properties of the samples. A user of the air quality monitor may interpret the measured properties of the samples as representing air quality for an entire room or an area of the room near the air quality monitor.

### SUMMARY

Example air quality monitors described herein each define a nonlinear flow path for sampled air, which enables the air quality monitor to have a relatively compact size. This nonlinear flow path occupies a relatively compact volume for use with a variety of air quality monitor form factors and permits sufficient flow through the air quality monitor for a relatively fast response time to changing air quality conditions.

In some examples, an air quality monitor includes a housing defining an inlet and an outlet, a fan, and one or more sensors disposed in the housing. The fan is configured to transport air along a nonlinear flow path from the inlet of the housing to the outlet of the housing. The one or more sensors are arranged along the nonlinear flow path and include a first sensor positioned upstream of the fan and a second sensor positioned downstream of the fan. The first sensor is configured to sense a first parameter of the air and the second sensor is configured to sense a second parameter of the air.

In some examples, a method for manufacturing an air quality monitor includes positioning a circuit board in a housing to define a first portion of a nonlinear flow path and a second portion of the nonlinear flow path downstream from the first portion of the nonlinear flow path. The circuit board includes an opening coupling the first and second portions of the nonlinear flow path. The circuit board is communicatively coupled to one or more sensors disposed in the housing and arranged along the nonlinear flow path. The method includes positioning a fan proximate to the opening in the circuit board. The fan is configured to transport air along the nonlinear flow path from an inlet of the housing to an outlet of the housing. The one or more sensors includes a first sensor positioned upstream of the fan and a second sensor disposed downstream of the fan. The first sensor is configured to sense a first parameter of the air and the second sensor is a second sensor positioned downstream of the fan and configured to sense a second parameter of the air.

In some examples, a method for monitoring air quality includes transporting, by a fan in an air quality monitor, air along a nonlinear flow path from an inlet of a housing of the air quality monitor to an outlet of the housing. The method includes sensing, by a first sensor positioned upstream of the fan, a first parameter of the air and sensing, by a second sensor positioned downstream of the fan, a second parameter of the air.

The details of one or more examples are set forth in the accompanying drawings and the description below. Other features, objects, and advantages will be apparent from the description and drawings, and from the claims.

### BRIEF DESCRIPTION OF DRAWINGS

FIG. 1A is a schematic diagram illustrating an example air quality monitor.

FIG. 1B is a schematic air flow path of the example air quality monitor of FIG. 1A.

FIG. 2A is a perspective view, semi-transparent diagram illustrating an example air quality monitor.

FIG. 2B is a front view, semi-transparent diagram illustrating the example air quality monitor of FIG. 2A.

FIG. 2C is a top view, semi-transparent diagram illustrating the example air quality monitor of FIG. 2A.

FIG. 2D is an inlet side view, semi-transparent diagram illustrating the example air quality monitor of FIG. 2A.

FIG. 2E is an outlet side view, semi-transparent diagram illustrating the example air quality monitor of FIG. 2A.

FIG. 3 is a perspective view diagram illustrating the example air quality monitor of FIG. 2A with an outer housing.

FIG. 4 is a flowchart illustrating an example method for manufacturing an air quality monitor.

FIG. 5 is a flowchart illustrating an example method for monitoring air quality using an air quality monitor.

### DETAILED DESCRIPTION

The disclosure describes air quality monitors having a relatively compact size. An air quality monitor may be positioned on a mounting surface, such as a table or wall, within a room, and may be configured to measure various properties of air within the room. To accommodate various aesthetically or spatially constrained designs, the air quality monitor defines a nonlinear flow path for sampled air. This nonlinear flow path occupies a relatively compact volume (e.g., relatively small largest dimension and/or relatively small volume) for use with a variety of form factors and permits sufficient flow through the air quality monitor for a relatively fast response time to changing air quality conditions.

Despite the relatively compact volume of the nonlinear flow path, the air quality monitor may measure various properties of the air at a relatively high accuracy by reducing an impact of various components of the air quality monitor on the air. For example, conditions near or within the air quality monitor may affect various properties of the air being sampled, such as temperature or composition, such that a sample of air of the nonlinear flow path may not represent the air in the room. In examples described herein, the air quality monitor is configured to draw air through an inlet at an adequate distance from the mounting surface (e.g., table or wall), such that the air may be less affected by properties of the mounting surface, such as surface temperature or directional flow. The air quality monitor is configured to measure various properties of the air according to a measurement sensitivity hierarchy, by first measuring various properties of air that are more likely to be affected by components of the air quality monitor, such that these prioritized measurements may be less affected by internal conditions of the air quality monitor (e.g., heat emitted from measurement components). The air quality monitor is configured to discharge the sampled air away from the inlet to reduce mixing of the sampled air with fresh air. In these various ways, air quality monitors discussed herein may be relatively compact, responsive, and/or accurate compared to air quality monitors that do not include a nonlinear flow path and do not include sensors arranged along a flow path in a measurement sensitivity hierarchy.

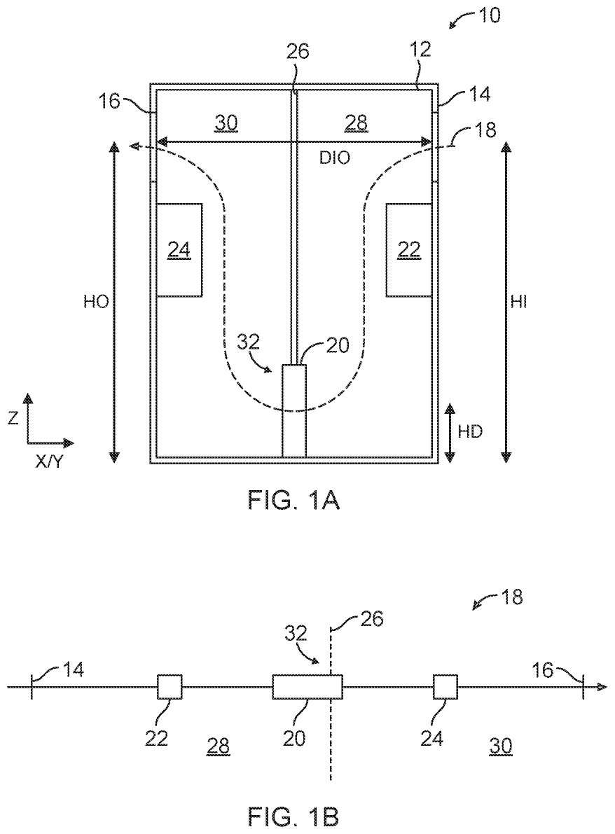

FIG. 1A is a schematic diagram illustrating an example air quality monitor 10. Air quality monitor 10 is configured to measure air quality for a variety of air compositions and conditions. For example, air quality may represent various properties of air, such as compositional properties (e.g., concentrations of air components) and/or physical properties (e.g., temperatures, pressures, and the like). As such, air quality monitor 10 is configured, such as by selection of components (e.g., sensing circuitry), to detect and/or measure the various properties of the air for various environmental conditions.

In some examples, air quality monitor 10 may be configured for use in a home environment. For example, air quality monitor 10 may be configured to measure air quality for compositional and physical properties commonly encountered in a living space, such as carbon dioxide concentration, carbon monoxide concentration, temperature, humidity, amount of dust particles, and the like. Air quality monitor 10 may be configured to quickly and accurately measure the air quality while also conforming to various aesthetic considerations. For example, air quality monitor 10 may be compatible with various form factors, such as compact form factors that enable a greater variety of designs than generally linear form factors, as will be described further below.

Air quality monitor 10 includes a housing 12. Housing 12 is configured to house (e.g., encase) and position components of air quality monitor 10 and define a volume for air to be analyzed. Housing 12 may be made from a variety of materials including, but not limited to, plastics, metals, ceramics, and combinations thereof. In some examples, housing 12 may have a relatively low thermal conductivity. For example, various components housed by housing 12 may be sensitive to changes in temperature, such that the relatively low thermal conductivity of the housing may reduce an amount of heat transferred to the various components in housing 12.

Housing 12 defines an inlet 14 and an outlet 16. Each of inlet 14 and outlet 16 may include one or more openings (e.g., voids in a plane defined by housing 12) in one or more walls of housing 12. Inlet 14 may be configured to receive air from an external environment near housing 12, while outlet 16 may be configured to discharge air to an external environment near housing 12. Housing 12 and, optionally, various components of air quality monitor 10 inside housing 12, define a nonlinear flow path 18 of air from inlet 14 to outlet 16. For example, components of air quality monitor 10 within housing 12 may be arranged to bound and/or direct flow along nonlinear flow path 18, such that a majority (e.g., greater than 50% and up to 100%) of air entering inlet 14 may proceed along nonlinear flow path 18 to outlet 16.

Nonlinear flow path 18 may be configured, such as through arrangement of components within housing 12, so that housing 12, and correspondingly air quality monitor 10, has a relatively compact size and/or shape. For example, an air quality monitor that includes a substantially linear flow path may be relatively limited to predominantly one-dimensional (e.g., an elongated cylinder or block) or predominantly two-dimensional (e.g., a flat block) shapes. In these cases, for an outer housing to cover the air quality monitor, such as for a certain aesthetic design, the outer housing may be either relatively large to accommodate the dominant dimension or dimensions, or relatively similar in shape to the air quality monitor.

In contrast to air quality monitors that define substantially linear flow paths for air, housing 12 may have a relatively compact design by having a relatively long nonlinear flow path 18 with respect to various dimensions of housing 12. For example, a high ratio of a length of nonlinear flow path 18 (e.g., a shortest distance of flow between inlet 14 and outlet 16) to a largest dimension of housing 12 may indicate a relatively compact design. In some examples, a ratio of a length of nonlinear flow path 18 to a largest dimension of housing 12 is greater than or equal to 1.5. A length of nonlinear flow path 18 can be, for example, the total length (when elongated) rather than the linear distance from end to end within housing 12. For comparison, a length of a linear flow path to a largest dimension of a housing of a linear air quality monitor may be close to 1.

In some examples, housing 12 may have a relatively compact design by having a relatively large volume with respect to various dimensions of housing 12. For example, housing 12 may be relatively compact by having relatively similar (e.g., within about 50%) width, length, and/or height, such that the summation of width, length, and height may be within about 5% of a minimum for an equivalent volume. In some examples, a ratio of a vertical component (e.g., normal to a plane of a mounting surface of housing 12) of the nonlinear flow path to a horizontal component (e.g., parallel with a plane of the mounting surface of housing 12) of the nonlinear flow path may be greater than or equal to about 1.5. For example, housing 12 may have a relatively small or compact basal area, such that nonlinear flow path 18 may primarily run vertically (e.g., normal to a mounting surface) through air quality monitor 10. In some examples, the ratio of the vertical component of the nonlinear flow path to the horizontal component of the nonlinear flow path (RAT) is represented by the equation RAT=((HI−HD)+(HO−HD))/DIO, wherein HI is a height of inlet 14 of housing 12, HD is a height of opening 32 of divider 26. HO is a height of outlet 16 of housing 12, and DIO is a horizontal distance between inlet 14 and outlet 16 of housing 12.

In some examples, housing 12 includes more than one chamber connected in series between inlet 14 and outlet 16. Each chamber may define a portion of nonlinear flow path 18. In the example of FIG. 1A, housing 12 includes a first chamber 28 and a second chamber 30 adjacent to first chamber 28. First chamber 28 defines a first portion of nonlinear flow path 18 and second chamber 30 defines a second portion of nonlinear flow path 18 downstream of the first portion of nonlinear flow path 18. The selection and/or arrangement of the chambers in housing 12 may enable air quality monitor 10 to have a relatively compact design. For example, while first chamber 28 and second chamber 30 may be arranged substantially in parallel (e.g., within parallel planes), first chamber 28 and second chamber 30 may be fluidically coupled in series, such that air flowing along nonlinear flow path 18 must first pass through first chamber 28 prior to passing through second chamber 30. As a result, housing 12 having more than one chamber may better control flow of air in a given volume than a housing that includes a common chamber of the same volume.

In some examples, housing 12 includes a divider 26 separating first chamber 28 and second chamber 30. Divider 26 includes an opening 32 fluidically coupling the first and second portions of nonlinear flow path 18. Divider 26 may be positioned relative to inlet 14 and/or outlet 16 to lengthen a path length of nonlinear flow path 18. For example, divider 26 may be positioned near a base (e.g., at or near the mounting surface) of housing 12, while inlet 14 and/or outlet 16 may be positioned near a vertical top (e.g., at or near the part furthest from the mounting surface) of housing 12.

Divider 26 has any suitable configuration and can be formed from any suitable material, such as a metal, a polymer, a ceramic, or combinations thereof. In some examples, divider 26 includes a circuit board communicatively coupled to various components of air quality monitor 10 and including, for example, control circuitry or the like configured to control an operation of air quality monitor 10. Divider 26 can be, for example, the circuit board or can be partially defined by the circuit board. In this way, the electronics of air quality monitor 10 can serve a dual purpose, which can further contribute to the compactness of air quality monitor 10 by eliminating the need for a separate structural component to function as divider 26.

Air quality monitor 10 includes one or more sensors disposed in housing 12. In the example of FIG. 1A, the one or more sensors include a first set of one or more sensors 22 and a second set of one or more sensors 24 (referred to collectively as “sensors 22 and 24”) located in first chamber 28 and second chamber 30, respectively. Sensors 22 and 24 may be arranged along nonlinear flow path 18, such that nonlinear flow path 18 may be configured to flow air through and/or across one or more surfaces or cavities of sensors 22 and 24 for sensing various parameters of the air. For example, air along nonlinear flow path 18 may interact with first set of sensors 22 in first chamber 28 prior to flowing into second chamber 30 to interact with second set of sensors 24.

Air quality monitor 10 includes at least one fan 20 configured to move air through housing 12 and configured to transport air along nonlinear flow path 18 from inlet 14 of housing 12 to outlet 16 of housing 12. Fan 20 defines a direction of air flow of nonlinear flow path 18. Fan 20 can be any suitable type of fan capable of generating air flow along nonlinear flow path 18. For example, fan 20 can include one or more of an axial flow fan, a centrifugal fan, or the like. In some examples, fan 20 is part of a sensor of air quality monitor 10, such as a particulate sensor (also referred to as a particulate matter sensor in some examples), while in other examples, fan 20 is separate from sensors of air quality monitor 10.

In some examples, fan 20 may be configured to generate a particular flow rate of air through nonlinear flow path 18. For example, a response time of sensors 22 and 24 to detect a change in properties of air may be related to an amount of time of the air to reach sensors 22 and 24. As such, a relatively high flow rate and/or a relatively low volume of nonlinear flow path 18 may reduce a response time of air quality monitor 10.

FIG. 1B is a linear schematic of nonlinear flow path 18 of the example air quality monitor of FIG. 1A. Although inlet 14, outlet 16, fan 20, sensors 22, 24, and divider 26 are shown in a linear arrangement in FIG. 1B, they are not linearly arranged in housing 12. The linear arrangement is only shown in FIG. 1B to illustrate the order in which air flows through nonlinear flow path 18 of housing 12.

First set of sensors 22 may be positioned upstream of fan 20, while second set of sensors 24 may be positioned downstream of fan 20. In operation, fan 20 may create a pressure differential between inlet 14 and outlet 16. As a result of this pressure differential, air may enter air quality monitor 10 through inlet 14, flow through and/or across first set of sensors 22, flow through fan 20, flow through and/or across second set of sensors 24, and leave air quality monitor 10 through outlet 16. As such, first set of sensors 22 may be configured to sense a one or more parameters of the air from nonlinear flow path 18 prior to the air passing through fan 20, second set of sensors 24, and other components of air quality monitor 10, while second set of sensors 24 may be configured to sense one or more parameters of the air from nonlinear flow path 18 after the air passes through components of air quality monitor 10.

In some examples, first set of sensors 22 may measure various parameters of the air that may be impacted by operation of second set of sensors 24, fan 20, or other components of air quality monitor 10. Certain surfaces or energy sources within air quality monitor 10 may affect whether the sample of the air sufficiently represents the quality of the air. For example, as air flows along nonlinear flow path 18, the air may receive heat from and/or intersect various components, such as a motor of fan 20, a circuit board, walls of housing 12, sensors 22 and 24, or any other components that may produce heat and/or intersect the air. This heat and/or intersection may change a temperature, humidity, and/or composition of particulates or liquids in the air. As a result, as the air flows along nonlinear flow path 18, various parameters of the air may deviate from the parameters of the air in the environment further along nonlinear flow path 18 the air travels. However, other parameters of the air, such as a composition of gases in air may be less likely to change in response to heat or a change in direction. To more accurately measure the air in the environment, parameters that may be relatively more impacted by the various components of air quality monitor 10 may be measured earlier in nonlinear flow path 18 than parameters that may be relatively less impacted by the various components of air quality monitor 10.

In some examples, first set of sensors 22 includes at least one of a temperature sensor, a humidity sensor, a particulate sensor disposed, and/or any other sensors that may be sensitive to a change in temperature or direction, disposed upstream of fan 20. In some examples, second set of sensors 24 includes at least one of a particulate sensor, a carbon dioxide sensor, a volatile organic compound (VOC) sensor, and/or any other sensors that may be relatively less sensitive to a change in temperature and/or direction, disposed downstream of first set of sensors 22 and/or fan 20.

In some examples, fan 20 is configured to discharge air through opening 32 in divider 26. For example, opening 32 in divider 26 may represent a choke point or a middle position of nonlinear flow path 18. To direct flow of air through opening 32 in divider 26 or more consistently control air flow through nonlinear flow path 18, fan 20 may be positioned proximate to opening 32 in divider 26, such that most or all air along nonlinear flow path 18 may pass through or proximate to fan 20 when flowing from first chamber 28 to second chamber 30. In comparison, a fan located near an inlet or an outlet of an air quality monitor may discharge air to a downstream portion of a flow path or draw air from an upstream portion of a flow path, respectively, with less directional control than a fan positioned proximate a choke point or a middle of a flow path. As such, fan 20 may better define and control the flow of air along nonlinear flow path 18.

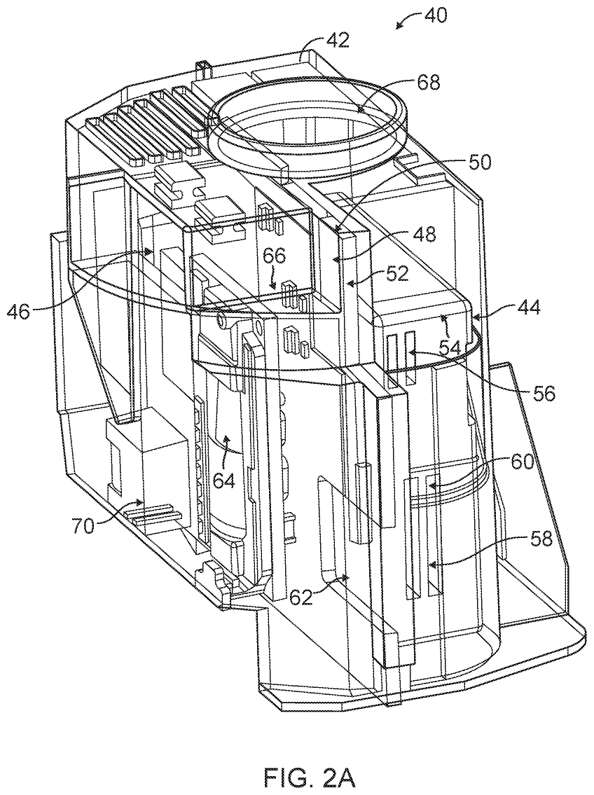

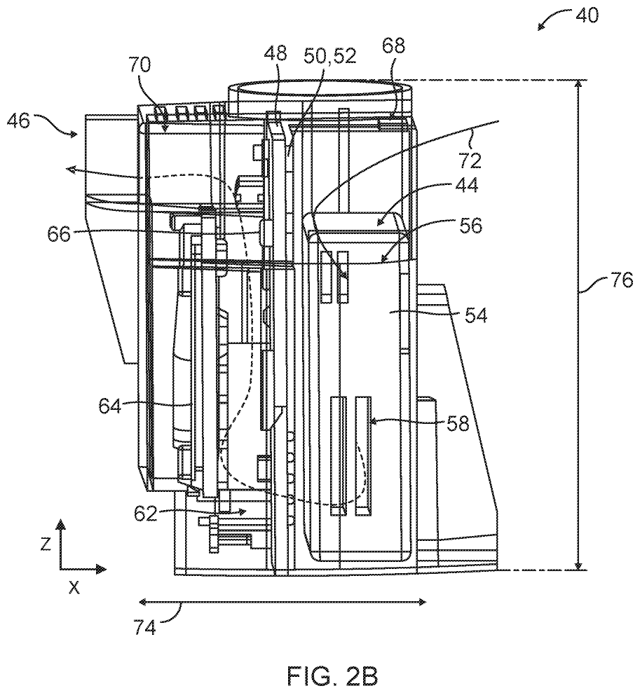

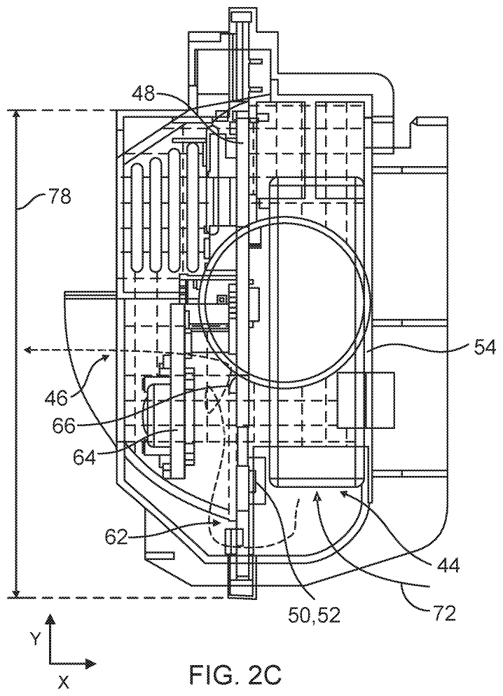

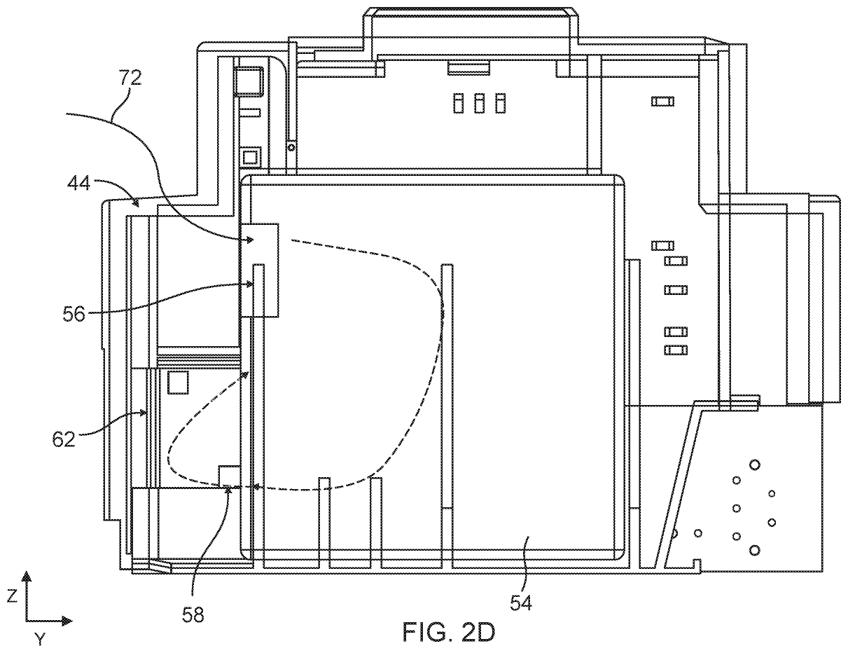

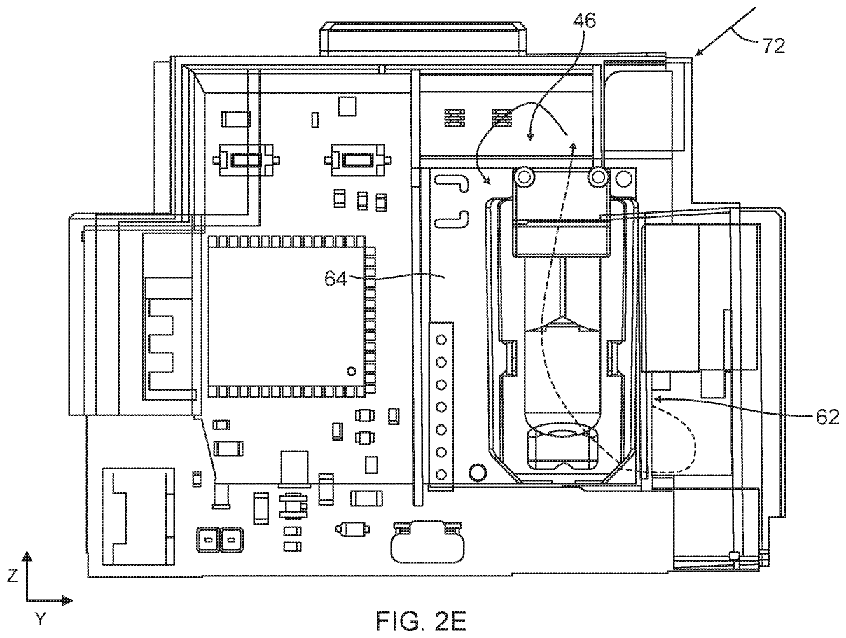

The principles of air quality monitor 10 described in FIG. 1A may be used to create air quality monitors that have a variety of functions and form factors. FIG. 2A is a perspective view, semi-transparent diagram illustrating an example air quality monitor 40. FIGS. 2B-2E represent various views illustrating a nonlinear flow path 72 of air through the example air quality monitor 40 of FIG. 2A. Nonlinear flow path 72 may be configured such that air quality monitor 40 may quickly and accurately detect various properties of air in a relatively compact and/or configurable form factor.

Air quality monitor 40 includes a housing 42 defining an outer boundary of air quality monitor 40. Housing 42 is an example of housing 12 of FIG. 1A and is configured to house components of air quality monitor 40 and define a volume for air to be analyzed. Housing 42 defines an inlet 44 and an outlet 46. Inlet 44 is configured to receive air from outside housing 42. Outlet 46 is configured to discharge air to outside housing 42. As illustrated in FIG. 2A, inlet 44 and/or outlet 46 may each be defined by an opening in a plane formed by the outer boundary of housing 42.

As described in FIG. 1A with respect to inlet 14 and outlet 16, inlet 44 and outlet 46 may be positioned such that air discharged from air quality monitor 40 through outlet 46 is separated from and not substantially mixed with air drawn into air quality monitor 40 through inlet 44. In some examples, separation between received and discharged air may be achieved by orienting a direction of inlet 44 away from outlet 46. In some examples, a direction of inlet 44 and a direction of outlet 46 (e.g., a direction from which air is generally received or discharged) may form an angle of at least 90 degrees, such as illustrated in FIG. 2A. In some examples, separation between received and discharged air may be achieved by separating inlet 44 and outlet 46 by a distance sufficient to substantially reduce mixing between received and discharged air. In some examples, a distance between inlet 44 of housing 42 and outlet 46 of housing 42 is greater than about 1 centimeter.

Housing 42 includes a first chamber 68 and a second chamber 70 adjacent to first chamber 68. Each chamber 68, 70 may define a portion of a nonlinear flow path 72 through air quality monitor 40. First chamber 68 defines a first portion of nonlinear flow path 72 and second chamber 70 defines a second portion of nonlinear flow path 72 downstream of the first portion of the nonlinear flow path 72. Housing 42 includes a divider 48 separating first chamber 68 and second chamber 70. In the example of FIG. 2A, at least a portion of divider 48 is a circuit board communicatively coupled to various components of air quality monitor 40. Divider 48 includes an opening 62 fluidically first chamber 68 and second chamber 70.

Air quality monitor 10 includes one or more sensors disposed in housing 42. In the example of FIG. 2A, the one or more sensors include a temperature sensor 50, a humidity sensor 52, a particulate sensor assembly 54, a carbon dioxide sensor 64, and a volatile organic compound (VOC) sensor 66. Sensors 50, 52, 54, 64, and 66 may be arranged along nonlinear flow path 72 and communicatively coupled to the circuit board of divider 48.

Temperature sensor 50 and humidity sensor 52 are disposed upstream of particulate sensor 54 near inlet 44. Temperature sensor 50 and humidity sensor 52 are configured to sense a temperature and humidity, respectively, of the air from nonlinear flow path 72. In some examples, temperature sensor 50 and humidity sensor 52 are part of a common integrated circuit positioned in housing 42. Air flowing through air quality monitor 40 along nonlinear flow path 72 may change in properties as the air passes from inlet 44 to outlet 46. For example, heat from various components within housing 42, such as the circuit board of divider 48 or sensors 54, 64, and 66, may generate heat and increase a temperature and/or decrease a humidity of the air, such that air exiting air quality monitor 40 through outlet 46 may be hotter and/or drier than air entering air quality monitor 40 through inlet 44. As such, temperature sensor 50 and humidity sensor 52 may be positioned on the circuit board near inlet 44, such that the temperature of the air measured may be less influenced by the other components of air quality monitor 40.

Particulate sensor assembly 54 is disposed downstream of temperature sensor 50 and humidity sensor 52. Particulate sensor assembly 54 is configured to sense a particulate presence and/or concentration in the air in the nonlinear flow path. A particulate concentration of the air may be less influenced by heat or changes in direction (e.g., from changes in direction) than a temperature or humidity of the air. In some examples, particulate sensor assembly 52 includes a housing that includes a particulate sensor inlet 56 and a particulate sensor outlet 58. In some examples, housing 42 includes a divider 60 configured to separate particulate sensor inlet 56 from particulate sensor outlet 58.

In the example of FIG. 2A, particulate sensor assembly 54 includes a fan integrated with a particulate sensor in the particulate sensor housing. Particulate sensor outlet 58 may be positioned proximate to opening 62 of divider 48, such that the fan may be configured to discharge air through opening 62 into second chamber 70. For example, particulate sensor assembly 54 may operate by drawing air through a particulate sensor inlet 56 through the particulate sensor and discharging the air out a particulate sensor outlet 58. In some examples, the particulate sensor may include a light source and an optical sensor to measure light scattered from particles in the air passing through the light beam of the light source. The light scattering may be influenced by both concentration of particulates and flow rate such that, to obtain a more accurate measurement, the flow rate of air may be controlled by the fan in particulate sensor assembly 54. In addition to discharging air through the particulate sensor, the fan of particulate sensor assembly 54 may be configured to transport air along nonlinear flow path 72 from inlet 44 to outlet 46. For example, the fan of particulate sensor assembly 54 may be capable of producing a sufficient flow rate (e.g., to achieve a desired residence or response time) of air through air quality monitor 40 to obtain a relatively fast response time, such as a residence time less than about four seconds. In this way, the same fan may be used to both measure a particulate presence and/or concentration and drive flow through air quality monitor 40, thereby reducing a size and/or complexity of air quality monitor 40. That is, in some examples, fan 20 (FIGS. 1A and 1B) of an air quality monitor can be the same fan as the fan of particulate sensor assembly 54.

In some examples, housing 42 may include a particulate sensor mount configured to secure the particulate sensor housing. For example, as explained above, particulate sensor assembly 54 may be a separate unit that includes a fan, a particulate sensor housing, and a particulate sensor. To accommodate this separate unit, the particulate sensor mount may be configured to receive particulate sensor assembly 54 into the mount and position particulate sensor assembly 54 to receive air from first chamber 68 and discharge air into second chamber 70.

Carbon dioxide sensor 64 and VOC sensor 66 are disposed downstream of temperature sensor 50 and humidity sensor 52. Carbon dioxide sensor 64 may be configured to sense a carbon dioxide presence and/or concentration. VOC sensor 66 may be configured to detect a volatile organic compound presence and/or concentration. Both carbon dioxide concentration and organic compound concentration may be relatively insensitive to heat or other conditions that may change various properties of the air as the air travels along the nonlinear flow path. As such, carbon dioxide sensor 64 and VOC sensor 66 may be positioned downstream of temperature sensor 50, humidity sensor 52, and particulate sensor assembly 54, and closer to outlet 46.

FIG. 2B illustrates a front view, semi-transparent diagram illustrating the example air quality monitor 40 of FIG. 2A. As illustrated in FIG. 2B, inlet 44 receives air from outside housing 42. Air entering inlet 44 may flow past temperature sensor 50 and humidity sensor 52 near inlet 44, such that a temperature and humidity measured by temperature sensor 50 and humidity sensor 52 may be relatively unaffected by components of air quality monitor 40 compared to measurements taken at other locations along nonlinear flow path 72.

In the example shown in FIGS. 2A-2E, air may flow along nonlinear flow path 72 into particulate sensor assembly inlet 56 due to a differential pressure created by a fan with particulate sensor assembly 54. The air may discharge from particulate sensor assembly outlet 58 and through opening 62 into second chamber 70. Air may flow relatively vertically through second chamber 70 past carbon dioxide sensor 64 and VOC sensor 66, and discharge from air quality monitor 40 through outlet 66, away from inlet 64. Air received by inlet 64 and discharged from outlet 66 may be separated by a portion of housing 42 and divider 48 to reduce mixing.

FIG. 2C is a top view, transparent diagram illustrating the example air quality monitor 40 of FIG. 2A. As illustrated in FIG. 2C, a volume from which air is drawn into inlet 44 is substantially separated from a volume to which air is discharged from outlet 46. A horizontal component of nonlinear flow path 72 may be relatively small compared to a vertical component of nonlinear flow path 72 illustrated in FIG. 2B.

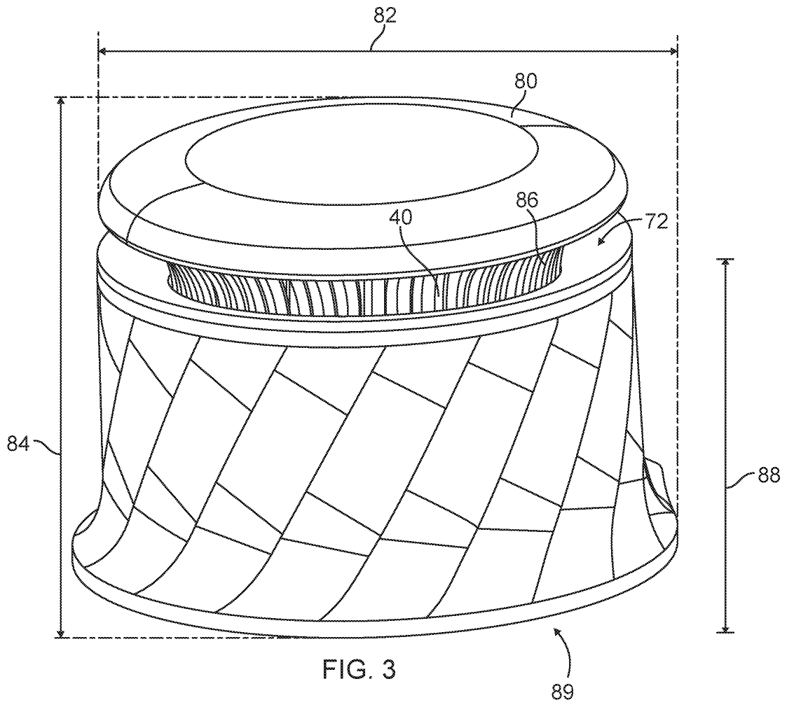

As illustrated in FIGS. 2B and 2C, housing 42 may have a width 74, height 76, and length 78 defining a general area and volume of occupied by housing 42. Housing 42 may be relatively compact. As one example, housing 42 may have a largest dimension of width 74 or length 78 less than twice as large as a smallest dimension of width or length, such that housing 42 may have a relatively compact basal area. For example, as will be described in FIG. 3, air quality monitor 40 may include an outer housing. In some examples, this outer housing may be symmetrical in at least two planes (e.g., circular or square), such that a more compact basal area may have a smaller corresponding side of the outer housing. As another example, housing 42 may have a largest dimension of width 74, height 76, and length 78 less than twice a smallest dimension of width 74, height 76, and length 78, such that housing 42 may have a relatively compact volume.

FIG. 2D is an inlet 44 side view, transparent diagram illustrating the example air quality monitor 40 of FIG. 2A, while FIG. 2E is an outlet 46 side view, transparent diagram illustrating the example air quality monitor 40 of FIG. 2A. As shown in FIGS. 2D and 2E, nonlinear flow path 72 may have a substantially vertical flow path while travelling through first chamber 68 and second chamber 70, respectively.

FIG. 3 is a perspective view diagram illustrating the example air quality monitor 40 of FIG. 2A with an outer housing 80 configured to house the components described with reference to FIGS. 2A-2E. For example, housing 42 may not be designed according to a particular form factor, but may rather be designed to be compatible with a variety of form factors. In such examples, housing 42 may be an inner housing, and air quality monitor 10 may include outer housing 80 around the inner housing 42. Outer housing 80 may define one or more openings 86 configured to permit air flow between inner housing 42 and an environment external to outer housing 80. As such, the one or more openings 86 may define intake of air above a mounting surface on which air quality monitor 40 is positioned. In some examples, the one or more openings 86 may be positioned at a height 88 of least about three centimeters from a base 89 of housing 42. In some examples, openings 86 are define by a mesh defining a plurality of pores or the like.

A compactness of inner housing 42 may enable outer housing 80 to be relatively compact. For example, a diameter 82 and a height 84 of outer housing 80 may be substantially similar (e.g., within about 50%). Outer housing 80 may include a variety of different designs including, but not limited to designs having a circular based, a squared base, a domed side profile, and the like.

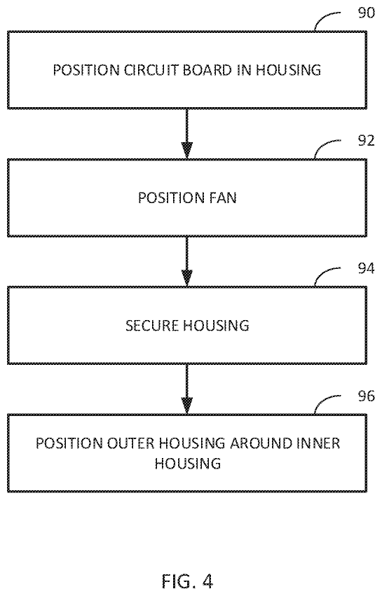

FIG. 4 is a flowchart illustrating an example method for manufacturing an air quality monitor. The technique of FIG. 4 will be described with respect to air quality monitor 40 of FIG. 2A; however, the technique of FIG. 4 may be used to form other air quality monitors. The method for manufacturing air quality monitor 10 may include forming housing 42. For example, housing 42 may include two or more components configured to be secured together to form housing 42. During positioning of various components of air quality monitor 40, housing 42 may remain disassembled.

The method may include forming the circuit board of divider 48. In some examples, forming the circuit board may include positioning one or more of sensors 50, 52, 64, or 66 on the circuit board of divider 48 and electrically and mechanically connecting the sensors to the circuit board. For example, rather than securing sensor 50, 52, 64, and 66 to the circuit board after divider 48 has been installed in air quality monitor 40, one or more of sensors 50, 52, 64, 66 may be communicatively coupled to the circuit board prior to positioning the circuit board in housing 42, such that sensors 50, 52, 64, 66 may be installed in a less space constrained environment and/or a separate facility in a less expensive manufacturing environment.

The method may include positioning divider 48 in at least a portion of housing 42 (90). Divider 48 may be positioned such that, once housing 42 is assembled, divider 48 may define a first portion of a nonlinear flow path and a second portion of the nonlinear flow path downstream from the first portion of the nonlinear flow path. The circuit board includes opening 62 coupling a first side of the circuit board that includes temperature sensor 50 and humidity sensor 52 with a second side of the circuit board that includes carbon dioxide sensor 64 and VOC sensor 66.

In some examples, the method includes positioning a fan proximate to opening 62 in the circuit board, such that the fan is configured to transport air along the nonlinear flow path from inlet 44 to outlet 46 (92). For example, the method may include positioning particulate sensor assembly 54, which includes a fan, in housing 42, such as on a particulate sensor mount, and communicatively coupling particulate sensor assembly 54 to the circuit board. Particulate sensor assembly 54 may be positioned proximate to opening 62 in the circuit board, such that particulate sensor assembly 54 may discharge air into second chamber 70.

In some examples, the method includes securing housing 42 to encase the components of air quality monitor 40 within housing 42 and define the nonlinear flow path through air quality monitor 40 (94). As a result, temperature sensor 50 and humidity sensor 52 may be positioned upstream of particulate sensor assembly 54 in first chamber 68, while carbon dioxide sensor 64 and VOC sensor 66 may be positioned downstream of particulate sensor assembly 54 in second chamber 70. In some examples, the method includes positioning outer housing 80 around air quality monitor 40 (100). For example, outer housing 80 may be positioned over housing 42.

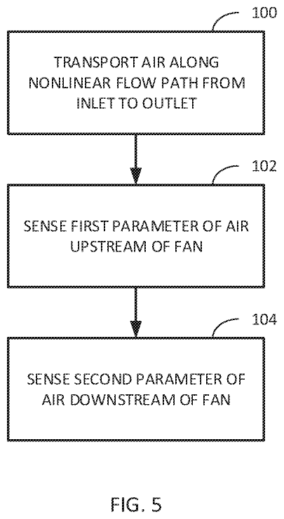

Various air quality monitors described herein may be used to monitor air quality of air FIG. 5 is a flowchart illustrating an example method for monitoring air quality using an air quality monitor. The technique of FIG. 5 will be described with respect to air quality monitor 10 of FIG. 1A and air quality monitor 40 of FIG. 2A; however, the technique of FIG. 5 may be used in other air quality monitors. The method for monitoring air quality may include transporting, by fan 20 in air quality monitor 10, air along nonlinear flow path 18 from inlet 14 of housing 12 of air quality monitor 10 to outlet 16 of housing 12 (110). For example, with respect to air quality monitor 40 of FIG. 2A, the method may include transporting, by particulate sensor assembly 54, air along the nonlinear flow path from inlet 44 to outlet 46. In some examples, air travelling along nonlinear flow path 18 may have a residence time less than about one second.

The method may include sensing, by first set of sensors 22 positioned upstream of fan 20, a first parameter of the air (112). For example, with respect to air quality monitor 40 of FIG. 2A, the method may include sensing, by temperature sensor 50 and humidity sensor 52 positioned upstream of particulate sensor assembly 54, a temperature and a humidity of the air. The method may include sensing, by second set of sensors 24 positioned downstream of fan 20, a second parameter of the air different from the first parameter (114). For example, with respect to air quality monitor 40 of FIG. 2A, the method may include sensing, by carbon dioxide sensor 64 and VOC sensor 66 positioned downstream of particulate sensor assembly 54, a carbon dioxide presence/concentration, and a VOC presence/concentration of the air.

The sensors described herein, including sensors 22 and 24 of FIGS. 1A and 1B and sensors 50, 52, 54, 64, and 66 of FIGS. 2A-2E, can have any suitable circuitry and components configured to provide the sensors with the desired function.

### EXAMPLES

### Example 1

In one example, an air quality monitor, comprising a housing defining an inlet and an outlet; a fan configured to transport air along a nonlinear flow path from the inlet of the housing to the outlet of the housing; and one or more sensors disposed in the housing and arranged along the nonlinear flow path, wherein the one or more sensors comprise: a first sensor positioned upstream of the fan and configured to sense a first parameter of the air; and a second sensor positioned downstream of the fan and configured to sense a second parameter of the air.

### Example 2

In some examples of the air quality monitor of example 1, the first parameter of the air is impacted by operation of the second sensor.

### Example 3

In some examples of the air quality monitor of example 1 or 2, a ratio of a length of the nonlinear flow path to a largest dimension of the housing is greater than 1.5.

### Example 4

In some examples of the air quality monitor of any of examples 1-3, the first sensor comprises a temperature sensor, and the second sensor comprises at least one of a particulate sensor, a carbon dioxide sensor, or a volatile organic compound (VOC) sensor.

### Example 5

In some examples of the air quality monitor of any of examples 1-4, wherein the first sensor comprises a temperature sensor and a humidity sensor, and wherein the second sensor comprises a particulate sensor, a carbon dioxide sensor, and a volatile organic compound (VOC) sensor disposed downstream of the temperature sensor and the humidity sensor.

### Example 6

In some examples of the air quality monitor of any of examples 1-5, the housing is an inner housing, and the air quality monitor further comprises an outer housing around the inner housing and defining one or more openings configured to permit air flow between the inner housing and an environment external to the outer housing.

### Example 7

In some examples of the air quality monitor of example 6, the one or more openings are positioned at least about three centimeters from a base of the outer housing.

### Example 8

In some examples of the air quality monitor of any of examples 1-7, the housing further comprises a first chamber defining a first portion of the nonlinear flow path; a second chamber adjacent to the first chamber and defining a second portion of the nonlinear flow path downstream of the first portion of the nonlinear flow path; and a divider separating the first chamber and the second chamber, wherein the divider includes an opening fluidically coupling the first and second portions of the nonlinear flow path.

### Example 9

In some examples of the air quality monitor of example 8, the air quality monitor further comprises a circuit board communicatively coupled to the one or more sensors, wherein the circuit board comprises at least a portion of the divider.

### Example 10

In some examples of the air quality monitor of example 8, the fan is configured to discharge air through the opening in the divider.

### Example 11

In some examples of the air quality monitor of example 8, a ratio of a vertical component of the nonlinear flow path to a horizontal component of the nonlinear flow path is greater than about 1.5.

### Example 12

In some examples of the air quality monitor of any of examples 1-11, wherein a distance between the inlet of the housing and the outlet of the housing is greater than about one centimeter.

### Example 13

In some examples of the air quality monitor of any of examples 1-12, the one or more sensors comprise a particulate sensor assembly, and the fan is integrated with a particulate sensor in a particulate sensor housing of the particulate sensor assembly.

### Example 14

In some examples of the air quality monitor of example 13, the housing comprises a particulate sensor mount configured to secure the particulate sensor assembly.

### Example 15

In some examples of the air quality monitor of example 13, the particulate sensor housing comprises a particulate sensor inlet and a particulate sensor outlet, and the housing comprises a divider configured to separate the particulate sensor inlet from the particulate sensor outlet.

### Example 16

In one example, a method for manufacturing an air quality monitor comprises positioning a circuit board in a housing to define a first portion of a nonlinear flow path and a second portion of the nonlinear flow path downstream from the first portion of the nonlinear flow path, wherein the circuit board includes an opening coupling the first and second portions of the nonlinear flow path, and wherein the circuit board is communicatively coupled to one or more sensors disposed in the housing and arranged along the nonlinear flow path; and positioning a fan proximate to the opening in the circuit board, wherein the fan is configured to transport air along the nonlinear flow path from an inlet of the housing to an outlet of the housing, wherein the one or more sensors comprise: a first sensor positioned upstream of the fan and configured to sense a first parameter of the air; and a second sensor positioned downstream of the fan and configured to sense a second parameter of the air.

### Example 17

In some examples of the method of example 16, the one or more sensors comprise a particulate sensor assembly, the fan is integrated with the particular sensor in a particulate sensor housing, and positioning the fan proximate to the opening in the circuit board further comprises positioning the particulate sensor assembly in the housing.

### Example 18

In one example, a method for monitoring air quality comprises transporting, by a fan in an air quality monitor, air along a nonlinear flow path from an inlet of a housing of the air quality monitor to an outlet of the housing; sensing, by a first sensor positioned upstream of the fan, a first parameter of the air; and sensing, by a second sensor positioned downstream of the fan, a second parameter of the air.

### Example 19

In some examples of the method of example 18, the first sensor comprises at least one of a temperature sensor or a humidity sensor, the second sensor comprises at least one of a particulate sensor, a carbon dioxide sensor, or a volatile organic compound (VOC) sensor, and the method further comprising sensing, by the at least one of the temperature sensor or the humidity sensor, a temperature or a humidity, respectively, of air in the flow path; and sensing, by the at least one of the particulate sensor, the carbon dioxide sensor, or the VOC sensor, a particulate concentration, carbon dioxide concentration, or VOC concentration, respectively, of the air after sensing the temperature and the humidity.

### Example 20

In some examples of the method of example 18 or 19, the nonlinear flow path has a residence time less than about four seconds.

Various examples have been described. These and other examples are within the scope of the following claims.

What is claimed is:

1. An air quality monitor, comprising:
a housing defining an inlet and an outlet;
a fan configured to transport air along a nonlinear flow path from the inlet of the housing to the outlet of the housing; and
one or more sensors disposed in the housing and arranged along the nonlinear flow path, wherein the one or more sensors comprise:
a first sensor positioned upstream of the fan and configured to sense a first parameter of the air; and
a second sensor positioned downstream of the fan and configured to sense a second parameter of the air.

2. The air quality monitor of claim 1, wherein the first parameter of the air is impacted by operation of the second sensor.

3. The air quality monitor of claim 1, wherein a ratio of a length of the nonlinear flow path to a largest dimension of the housing is greater than 1.5.

4. The air quality monitor of claim 1, wherein the first sensor comprises a temperature sensor, and wherein the second sensor comprises at least one of a particulate sensor, a carbon dioxide sensor, or a volatile organic compound (VOC) sensor.

5. The air quality monitor of claim 1, wherein the first sensor comprises a temperature sensor and a humidity sensor, and wherein the second sensor comprises a particulate sensor, a carbon dioxide sensor, and a volatile organic compound (VOC) sensor disposed downstream of the temperature sensor and the humidity sensor.

6. The air quality monitor of claim 1, wherein the housing is an inner housing, and wherein the air quality monitor further comprises an outer housing around the inner housing and defining one or more openings configured to permit air flow between the inner housing and an environment external to the outer housing.

7. The air quality monitor of claim 6, wherein the one or more openings are positioned at least about three centimeters from a base of the outer housing.

8. The air quality monitor of claim 1, wherein the housing further comprises:
a first chamber defining a first portion of the nonlinear flow path;
a second chamber adjacent to the first chamber and defining a second portion of the nonlinear flow path downstream of the first portion of the nonlinear flow path; and
a divider separating the first chamber and the second chamber, wherein the divider includes an opening fluidically coupling the first and second portions of the nonlinear flow path.

9. The air quality monitor of claim 8, further comprising a circuit board communicatively coupled to the one or more sensors, wherein the circuit board comprises at least a portion of the divider.

10. The air quality monitor of claim 8, wherein the fan is configured to discharge air through the opening in the divider.

11. The air quality monitor of claim 8, wherein a ratio of a vertical component of the nonlinear flow path to a horizontal component of the nonlinear flow path is greater than about 1.5.

12. The air quality monitor of claim 1, wherein a distance between the inlet of the housing and the outlet of the housing is greater than about one centimeter.

13. The air quality monitor of claim 1, wherein the one or more sensors comprise a particulate sensor assembly, and wherein the fan is integrated with a particulate sensor in a particulate sensor housing of the particulate sensor assembly.

14. The air quality monitor of claim 13, wherein the housing comprises a particulate sensor mount configured to secure the particulate sensor assembly.

15. The air quality monitor of claim 13,
wherein the particulate sensor housing comprises a particulate sensor inlet and a particulate sensor outlet, and
wherein the housing comprises a divider configured to separate the particulate sensor inlet from the particulate sensor outlet.

16. A method for manufacturing an air quality monitor, the method comprising:
positioning a circuit board in a housing to define a first portion of a nonlinear flow path and a second portion of the nonlinear flow path downstream from the first portion of the nonlinear flow path, wherein the circuit board includes an opening coupling the first and second portions of the nonlinear flow path, and wherein the circuit board is communicatively coupled to one or more sensors disposed in the housing and arranged along the nonlinear flow path, and
positioning a fan proximate to the opening in the circuit board, wherein the fan is configured to transport air along the nonlinear flow path from an inlet of the housing to an outlet of the housing,
wherein the one or more sensors comprise:
a first sensor positioned upstream of the fan and configured to sense a first parameter of the air; and
a second sensor positioned downstream of the fan and configured to sense a second parameter of the air.

17. The method of claim 16,
wherein the one or more sensors comprise a particulate sensor assembly,
wherein the fan is integrated with the particular sensor in a particulate sensor housing, and
wherein positioning the fan proximate to the opening in the circuit board further comprises positioning the particulate sensor assembly in the housing.

18. A method for monitoring air quality, the method comprising:
transporting, by a fan in an air quality monitor, air along a nonlinear flow path from an inlet of a housing of the air quality monitor to an outlet of the housing;
sensing, by a first sensor positioned upstream of the fan, a first parameter of the air; and
sensing, by a second sensor positioned downstream of the fan, a second parameter of the air.

19. The method of claim 18, wherein the first sensor comprises at least one of a temperature sensor or a humidity sensor, and the second sensor comprises at least one of a particulate sensor, a carbon dioxide sensor, or a volatile organic compound (VOC) sensor, the method further comprising:
sensing, by the at least one of the temperature sensor or the humidity sensor, a temperature or a humidity, respectively, of air in the flow path; and
sensing, by the at least one of the particulate sensor, the carbon dioxide sensor, or the VOC sensor, a particulate concentration, carbon dioxide concentration, or VOC concentration, respectively, of the air after sensing the temperature and the humidity.

20. The method of claim 18, wherein the nonlinear flow path has a residence time less than about four seconds.
